(19) United States
(12) Patent Application Publication
(10) Pub. No.: US 2025/0311917 A1
(43) Pub. Date: Oct. 9, 2025

(54) METHOD AND APPARATUS FOR ACCURATELY MANIPULATING A PORTION OF A MEDICAL DEVICE

(71) Applicant: TANGENT ENDOSCOPY, LLC, Camden, DE (US)

(72) Inventors: Scott Richard ARP, Miami, FL (US); Joseph Paul AGUILA, Fort Lauderdale, FL (US); Berry LAMY, Boynton Beach, FL (US); Angel MENDOZA, Tamarac, FL (US)

(21) Appl. No.: 18/991,593

(22) Filed: Dec. 22, 2024

Related U.S. Application Data
(60) Provisional application No. 63/574,596, filed on Apr. 4, 2024.

(51) Int. Cl. A61B 1/005; A61M 25/01

(52) U.S. Cl. CPC A61B 1/0057; A61B 1/0052; A61M 25/0136; A61M 25/0147

(57) ABSTRACT

A method and apparatus for tensioning a control wire in a medical device comprising an actuator, adapted to receive an input rotary force, for converting the rotary force into linear motion. A plurality of control wires are attached to the actuator and to a controllable portion of the medical device. The control wires are linearly moved by the actuator to manipulate the controllable portion of the medical device.

### RELATED APPLICATION

This application claims benefit to U.S. Provisional Patent Application Ser. No. 63/574,596 filed 4 Apr. 2024 entitled “Method and Apparatus for Accurately Manipulating a Portion of a Medical Device,” which is hereby incorporated herein by reference in its entirety.

### BACKGROUND

### Field

Embodiments of the present invention generally relate to medical devices and, in particular, to a method and apparatus for manipulating a portion of a medical device.

### Description of the Related Art

A typical medical device, such as an endoscope, comprises an actuator and a substantially hollow, flexible shaft extending from the actuator. A portion of the shaft (typically, the tip) carries medical instrumentation for diagnosis and/or treatment of a medical condition. The actuator manipulates at least one control wire that extends, along the inside of the shaft, from the actuator to a portion of the medical device, for example, the distal end of the shaft. The actuator generally comprises a take-up wheel coupled to a control knob or slider. The at least one control wire is attached to the take-up wheel. As the knob or slider is manually moved, the take-up wheel rotates and more or less control wire wraps around the wheel. As the control wire is taken-up by the wheel, the distal end of the flexible shaft is curved in a specific direction depending on the attachment point of the wire to the flexible shaft.

If the medical device comprises two wires, the wires are attached to opposite sides of the wheel and manipulation of the actuator moves the distal end of the shaft left or right, or up or down. If the medical device comprises four wires. The wires are attached to at least two independently controllable take-up wheels and manipulation of the actuator moves the distal end of the shaft left, right, up, or down, i.e., three-dimensional manipulation.

As the actuator is manipulated, the wire that is tensioned by the take-up wheel moves the distal end in a particular direction. The wires that are not tensioned will have slack in them. Upon changing direction of the manipulator, the slack must be taken-up before the distal end of the shaft will move. Such slack causes a lack of end motion for a time period until the slack is taken-up. This slack induced hysteresis can result in inaccurate positioning of the endoscope distal end and incorrect positioning of a medical instrument located at the distal end.

Therefore, there is a need for a method and apparatus for accurately manipulating a portion of a medical device.

### SUMMARY

A method and apparatus for accurately manipulating a tip of a medical device is provided substantially as shown in and/or described in connection with at least one of the figures, as set forth more completely in the claims.

Various features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

So that the manner in which the above recited features of the present invention can be understood in detail, a particular description of the invention, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 depicts a perspective view of a medical device in accordance with at least one embodiment of the invention;

FIG. 2A and 2B respectively depict a first half and a second half of the actuator of the medical device of FIG. 1 in accordance with at least one embodiment of the invention;

FIG. 3 depicts the actuator manipulating a control wire in accordance with at least one embodiment of the invention;

FIG. 4A and 4B respectively depict a top plan view of a wire manipulator drive gear within the actuator and a side view of the wire manipulator drive gear in accordance with at least one embodiment of the invention;

FIG. 5 depicts a perspective view of a wire manipulator in accordance with at least one embodiment of the invention;

FIG. 6 depicts a top view of the wire manipulator of FIG. 5 in accordance with at least one embodiment of the invention;

FIG. 7 depicts plan view of the wire manipulator of FIG. 5 in accordance with at least one embodiment of the invention;

FIG. 8 depicts side view of the wire manipulator of FIG. 5 in accordance with at least one embodiment of the invention;

FIG. 9 depicts a side cross-sectional view of a portion of the medical device to which control wires are attached in accordance with at least one embodiment of the invention; and

FIG. 10 depicts an end cross-sectional view of the portion along lines 9-9 in FIG. 9 in accordance with at least one embodiment of the invention.

### DETAILED DESCRIPTION

Embodiments of the present invention include a method and apparatus for accurately manipulating a portion of a medical device, such as an endoscope. The medical device comprises an actuator coupled to a substantially hollow, flexible shaft. The actuator is coupled to a plurality of control wires that extend internal of the shaft from the actuator to a controllable portion of the shaft, e.g., at a location near a distal end (or tip) of the shaft. Manipulation of the actuator pulls on the control wires and causes the distal end of the shaft to move in a specific direction. In one embodiment of the invention, the actuator comprises a first half and a second half. Each half has a controller, e.g., a lever, a knob, servo, motor, solenoid, etc. through which input force is applied to the actuator. Each controller manipulates the controllable portion of the medical device in a different plane where the planes are typically perpendicular to one another. As such, through adjusting the controllers, the controllable portion may be moved in three dimensions.

The actuator utilizes a manipulator mechanism that ensures the plurality of control wires never have slack in them. Consequently, manipulation of the controllers immediately causes accurate movement of the controllable portion.

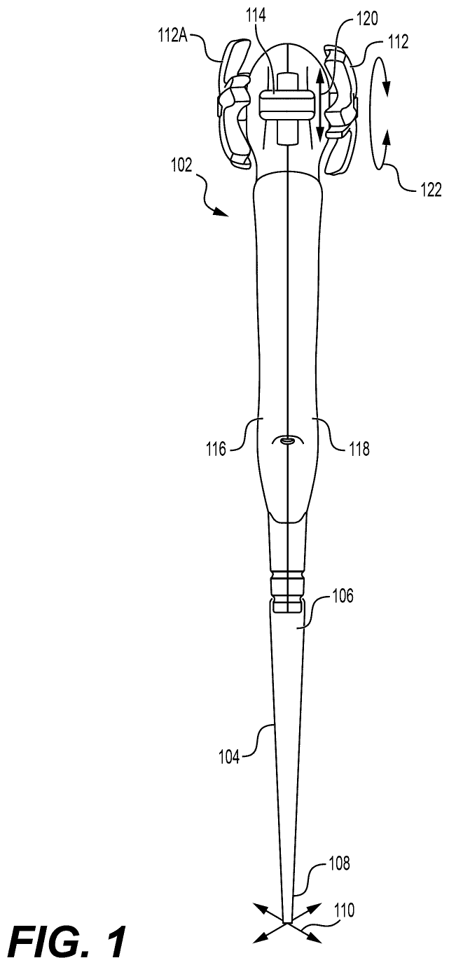

FIG. 1 depicts a perspective view of a medical device, e.g., an endoscope 100, in accordance with at least one embodiment of the invention. The endoscope 100 comprises an actuator 102 coupled to a hollow flexible shaft 104. The shaft 104 comprises a proximal end 106 coupled to the actuator 102 and a controllable portion such as a distal end 108 (also referred to as a tip) of the shaft 104 that is directionally manipulated by the actuator 102 (as indicated by arrows 110). In other embodiments, the controllable portion may be part way along the flexible shaft 104. The actuator 102 has two halves: first half 116 and second half 118. In one embodiment, the actuator 102 comprises two controllers: a knob 112 and a lever 114 that are manually manipulated to move the controllable portion of the shaft 104. In some embodiments, a second knob 112A may be included to allow ambidextrous use of the actuator 102. In the depicted embodiment, the lever 114 is located in first half 116 and slides in a manner as indicated by arrow 120 to manipulate the internal control wires. In the depicted embodiment, the knob 112 is located on the second half 118 and rotates as indicated by arrow 122 to manipulate the internal control wires. In other embodiments, the actuator 102 comprises automated controllers such as servos, stepper motors, solenoids, and the like (not shown) that can be electronically manipulated to move the controllable portion of the shaft 104.

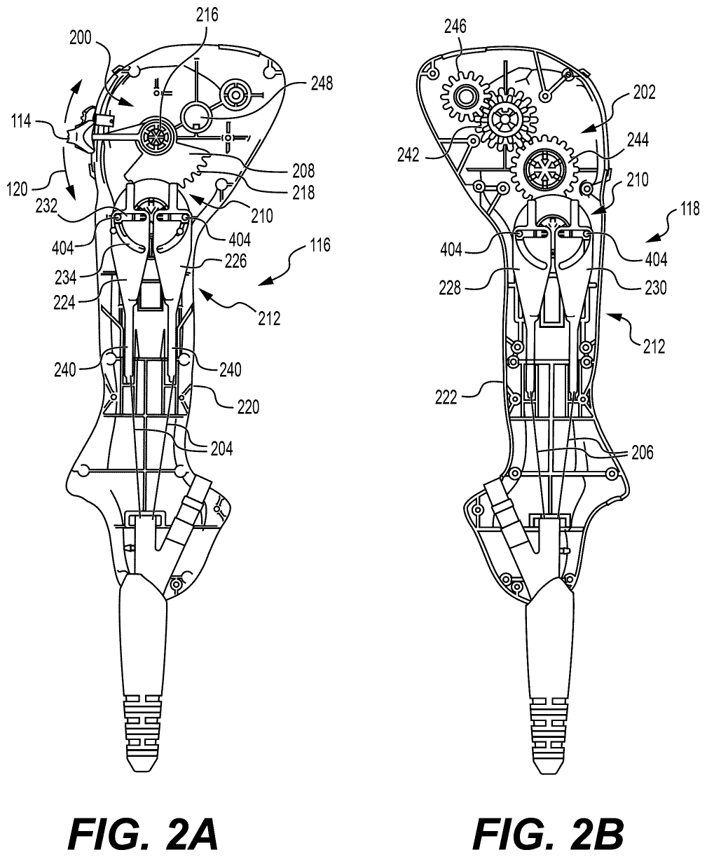

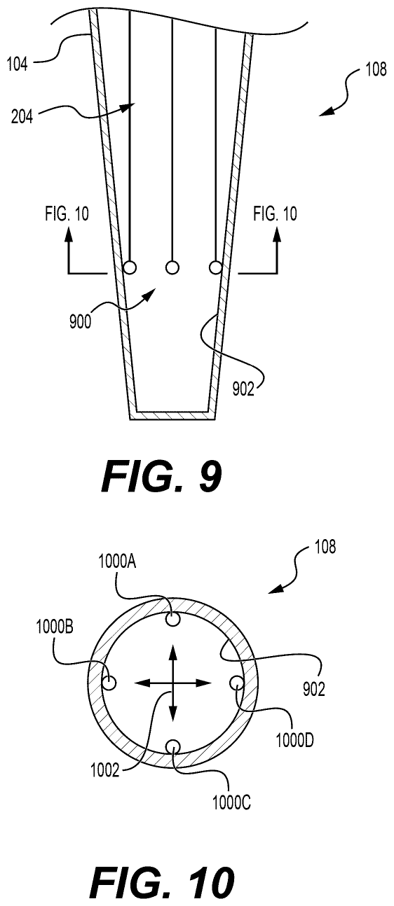

FIG. 2A and 2B respectively depict a first half 116 and a second half 118 of the actuator 102 of the endoscope 100 of FIG. 1 in accordance with at least one embodiment of the invention. Each half 116 and 118 comprise housings 220 and 222 that support independently operable control wire actuator mechanisms 200 and 202. When fully assembled, the two housings 220 and 222 are attached to one another using screws, screws and nuts, rivets, snap fittings, compression fittings, welding, adhesive, or a combination of such means for attaching the two housing halves 220 and 222. The means for attaching the halves are well known by those skilled in the art and, for clarity, are not shown in the drawings. Each mechanism 200 and 202 manipulates a pair of control wires 204 and 206. In one exemplary embodiment, the wires 204 are attached to the controllable portion in a first plane and the wires 206 are attached in a second plane, where the first and second planes are perpendicular to one another. The wires 204 and 206 are typically attached to the shaft near its distal end as described in detail with respect to FIGS. 9 and 10 below.

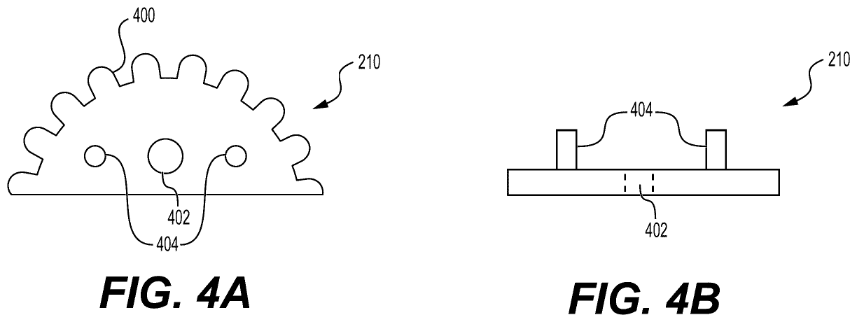

The lever mechanism 200 comprises the lever 114, a drive gear 208, a wire manipulator drive gear 210, and a wire manipulator 212. The drive gear 208 is coupled to the lever 114 such that, when the lever is moved along arrow 120 to apply an input force, the gear 208 is rotated (i.e., a rotary input force is applied to the actuator). The gear 208 comprises an axis of rotation 216 and teeth 218. The teeth 218 of the drive gear 208 interact with and drive the wire manipulator drive gear 210 (hidden behind the manipulator 212 but shown in FIGS. 4A and 4B). The drive gear 208 rotates the wire manipulator drive gear 210 to drive the manipulator 212 that pulls upwards on one or the other wire 204 depending on the direction of the lever movement.

FIG. 4A and 4B respectively depict a top plan view of the wire manipulator drive gear 210 and a side view of the wire manipulator drive gear 210 in accordance with at least one embodiment of the invention. The gear 210 is a half gear comprising teeth 400, an axis of rotation 402 and two spaced-apart posts 404. The teeth 218 of the drive gear 208 interact with the teeth 400 of the wire manipulator drive gear 210 to rotate the gear 210 about the axis 402. The rotation moves the posts 404 about the axis 402. The posts 404 interact with the wire manipulator 212.

The gear ratio (relative number and spacing of teeth on each gear) determines the amount of input force that is translated into linear pulling force on the wire 204. The wire manipulator 212 manipulates a single wire 204/206 without moving (i.e., pushing) the opposite wire.

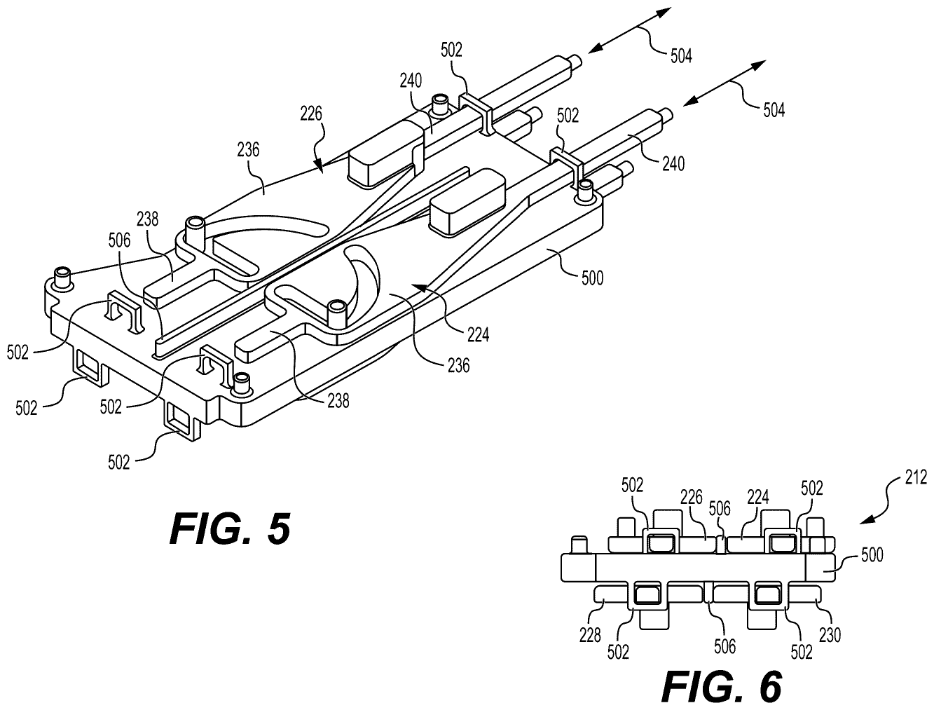

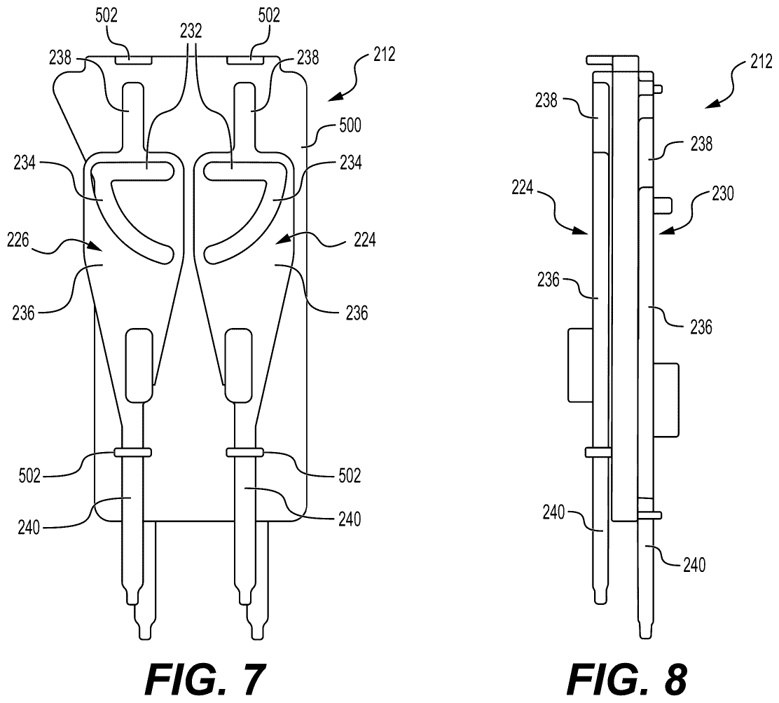

FIGS. 5, 6, 7, and 8 respectively depict a perspective view, a top view, a plan view, and a side view of the wire manipulator 212 in accordance with at least one embodiment of the invention. To understand the structure of the manipulator 212, FIGS. 2A, 2B, 5, 6, 7, and 8 should be viewed simultaneously with the following description. The manipulator 212 comprises a pair of sliders 224 and 226 that are slidably mounted to a platform 500 (not shown in FIGS. 2A and 2B). The manipulator 212 is assembled as a modular mechanism that resides between the two halves 116 and 118 such that the platform 500 has a first side that supports a first pair of sliders 224 and 226 for half 116 and a second side that supports a second pair of sliders 228 and 230 for half 118. In the exemplary embodiment, each slider 224, 226, 228, 230 comprises a body 236, an upper guide portion 238 and a lower guide portion 240.

The pair of sliders 224 and 226 (and slider pair 228 and 230) are retained upon the platform 500 by a retainer 502 that, in one embodiment comprises four brackets (or other form of guide such as pins, rails, etc.) that retain the upper and lower guide portions 238 and 240 and facilitate linear motion of the sliders 224 and 226. The retainer 502 allow the sliders 224, 226, 228, 230 to linearly move along the platform (see arrow 504). In addition, the platform 500 includes a guiderail 506 located between the sliders 224 and 226 (as well as between 228 and 230) to ensure the sliders move in a linear fashion. The combination of the manipulator drive gear 210 and the manipulator 212 convert rotational movement of the gear 210 into linear movement of the sliders 224, 226, 228, and 230 and wires 204, 206. Each slider 224, 226, 228, and 230 comprises a horizontal slot 232 and an arcuate slot 234 with an end of the horizontal slot 232 connected to an end of the arcuate slot 234. When assembled, each post 404 of the manipulator drive gear are positioned in the slots 232 and 234. The neutral position is to have the post 404 located at the junction of the horizontal slot 232 and the arcuate slot 234. As the manipulator drive gear 210 rotates, one post 404 slides into the horizontal slot 232 and the other post 404 slides into the arcuate slot 234. The post 404 in the horizontal slot 232 moves its associated slider 224, 226, 228, and 230 upwards to pull on the wire 204 that is attached to the slider 224, 226, 228, and 230. The post 404 in the arcuate slot 234 slides along the arcuate slot 234 without moving the associated slider 224, 226, 228, and 230. The wire 204, 206 is attached to the end of the lower guide portion 240 of each slider 224, 226, 228, and 230. In one embodiment, the wire is attached with a ferrule (not shown) crimped onto the end of the wire 204, 206 positioned in a slot (not shown) that retains the ferrule. In other embodiments, the wire may be attached using other techniques such as welding, tying, adhesive, screw, etc.

The actuator mechanism 202 of the second half 118 is driven by at least one knob (knob 112 of FIG. 1). The actuator mechanism 202 comprises a first drive gear 242, a second drive gear 244, and the manipulator drive gear 210. If a second knob 112A is to be included, the actuator 202 includes a coupling gear 246 that couples the rotation on one knob 112 to the other knob 112A. The second knob 112A would have a set of gears (not shown) in the second half 116 driving the second knob 112A via shaft 248. Having a second knob 112A facilitate ambidextrous use of the medical device 100. The rotational input force applied by either knob 112, 112A (or any robotic input force generator such as a servo, motor, solenoid, etc.) is translated by the manipulator 212 into a linear force upon the wires 206. The number of teeth and spacing of the teeth upon the gears 242, 244, 210 and 246 establishes the force translation ratio for the input force to the force applied to the wires 206.

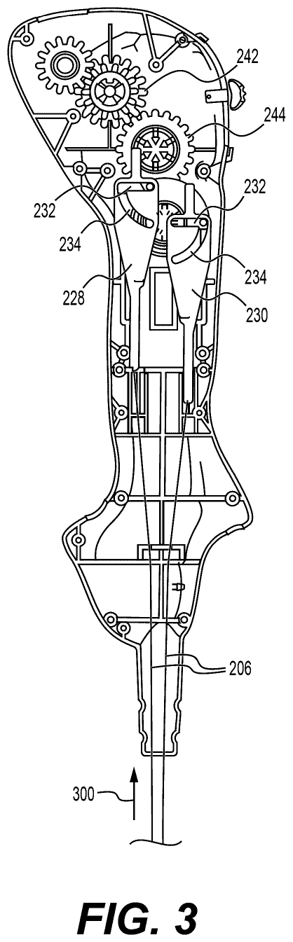

FIG. 3 depicts the actuator 202 manipulating a control wire 204 in accordance with at least one embodiment of the invention. In operation, as the knob 112 is turned counterclockwise (when looking at the knob) to turn the first drive gear 242 clockwise, the second drive gear 244 turns counterclockwise and the manipulator drive gear 210 rotates clockwise. The clockwise rotation of gear 210 moves the post 404 into the horizontal slot 232 and the other post 404 into the arcuate slot 234. Travel in the horizontal slot 232 moves the slider 228 upward to pull on the wire 204 as shown by arrow 300. Simultaneously, the post 404 moving in the arcuate slot 234 does not move the slider 230 (i.e., the slider 230 remains stationary). As the wire is pulled upward, the controllable portion (e.g., the shaft distal end) moves in the direction of the wire connection location within the shaft. When the knob 112 is rotated in the opposite direction, the other wire 204 is pulled by slider 230 and slider 228 is stationary. The other half 116 works in exactly the same manner when the lever is manipulated. Manipulation of both the lever and knob (or any robotic input force generator) accurately manipulates the controllable portion of the medical device in three-dimensions.

FIG. 9 depicts a side cross-sectional view of an exemplary controllable portion of the medical device (e.g., an endoscope shaft distal end 108) in accordance with at least one embodiment of the invention. FIG. 10 depicts an end cross-sectional view along lines 10-10 in FIG. 9 of the controllable portion in accordance with at least one embodiment of the invention. The distal end 108 generally forms the tip of the hollow, flexible shaft 104 in FIG. 1 where medical instrumentation may be mounted. Within the shaft, the ends 700 of the control wires 204 and 206 are attached to the inside surface 902 of the shaft 104. As shown in FIG. 10, for a four-wire endoscope, the wires 204 and 206 are attached at 90-degree locations 1000A, 1000B, 1000C, and 1000D around the circumference of the inner surface 902 of the shaft 104. Linear motion of the wires 204 and 206 causes the controllable portion (e.g., tip) to be moved in a particular direction represented by the arrows 1002.

Here multiple examples have been given to illustrate various features and are not intended to be so limiting. Any one or more of the features may not be limited to the particular examples presented herein, regardless of any order, combination, or connections described. In fact, it should be understood that any combination of the features and/or elements described by way of example above are contemplated, including any variation or modification which is not enumerated, but capable of achieving the same. Unless otherwise stated, any one or more of the features may be combined in any order.

As above, figures are presented herein for illustrative purposes and are not meant to impose any structural limitations, unless otherwise specified. Various modifications to any of the structures shown in the figures are contemplated to be within the scope of the invention presented herein. The invention is not intended to be limited to any scope of claim language.

Where conditional language is used, including, but not limited to, “can,” “could,” “may” or “might,” it should be understood that the associated features or elements are not required. As such, where conditional language is used, the elements and/or features should be understood as being optionally present in at least some examples, and not necessarily conditioned upon anything, unless otherwise specified.

Where lists are enumerated in the alternative or conjunctive (e.g., one or more of A, B, and/or C), unless stated otherwise, it is understood to include one or more of each element, including any one or more combinations of any number of the enumerated elements (e.g. A, AB, AC, ABC, ABB, etc.). When “and/or” is used, it should be understood that the elements may be joined in the alternative or conjunctive.

While the foregoing is directed to embodiments of the present invention, other and further embodiments of the invention may be devised without departing from the basic scope thereof, and the scope thereof is determined by the claims that follow.

1. Apparatus for manipulating a controllable portion of a medical device comprising:
an actuator, adapted to receive an input rotary force, for converting the rotary force into linear motion; and
a plurality of control wires, attached to the actuator and to the controllable portion of the medical device, that are independently linearly moved by the actuator to manipulate a position of the controllable portion of the medical device.

2. The apparatus of claim 1, wherein the input rotary force is generated through movement of at least one of a lever or a knob.

3. The apparatus of claim 2, wherein the at least one lever or knob is coupled to a manipulator drive gear which, when the input rotary force is applied, the actuator moves a guide wire without moving any other guide wires in the plurality of guide wires.

4. The apparatus of claim 3, wherein the actuator comprises:
a wire manipulator comprising a pair of sliders that are slidably mounted to a platform;
each control wire in the plurality of control wires is coupled to one of the sliders in the pair of sliders; and
the pair of sliders are coupled to the manipulator drive gear.

5. The apparatus of claim 4, wherein the manipulator drive gear comprises a pair of spaced apart posts and each post interacts with a slot formed in each slider in the pair of sliders.

6. The apparatus of claim 5, wherein the slot comprises a horizontal slot and an arcuate slot.

7. The apparatus of claim 5, wherein each slider comprises an upper and lower guide portion and the platform comprises a retainer that retains the sliders upon the platform while allowing the sliders to move linearly relative to the platform.

8. The apparatus of claim 4, wherein the platform has a first side and a second side and a first pair of sliders is slidably mounted to the first side and a second pair of sliders is slidably mounted to the second side.

9. A method of operation of a medical device comprising:
receiving input rotary force;
converting the input rotary force into a linear motion; and
coupling the linear motion to at least one control wire that is coupled to a controllable portion of the medical device to manipulate a position of the controllable portion of the medical device.

10. The method of claim 9, wherein the input rotary force is produced by at least one of a knob or lever.

11. The method of claim 9, the converting further comprises using a wire manipulator to linearly move at least one control wire, while maintaining at least one other control wire in a stationary position.

12. A medical device comprising:
an actuator, adapted to receive an input rotary force, for converting the rotary force into linear motion;
a hollow flexible shaft, coupled to the actuator, having a controllable position proximate a distal end of the hollow flexible shaft; and
a plurality of control wires, attached to the actuator and to the controllable portion, that is linearly moved by the actuator to manipulate a position of the controllable portion of the medical device.

13. The medical device of claim 12, wherein the input rotary force is generated through movement of at least one of a lever or a knob.

14. The medical device of claim 13, wherein the at least one lever or knob is coupled to a manipulator drive gear which, when the input rotary force is applied, the actuator moves a guide wire without moving any other guide wires in the plurality of guide wires.

15. The medical device of claim 14, wherein the actuator comprises:
a wire manipulator comprising a pair of sliders that are slidably mounted to a platform;
each control wire in the plurality of control wires is coupled to one of the sliders in the pair of sliders; and
the pair of sliders are coupled to the manipulator drive gear.

16. The medical device of claim 15, wherein the manipulator drive gear comprises a pair of spaced apart posts and each post interacts with a slot formed in each slider in the pair of sliders.

17. The medical device of claim 16, wherein the slot comprises a horizontal slot and an arcuate slot.

18. The medical device of claim 16, wherein each slider comprises an upper and lower guide portion and the platform comprises a retainer that retains the sliders upon the platform while allowing the sliders to move linearly relative to the platform.

19. The medical device of claim 15, wherein the platform has a first side and a second side and a first pair of sliders is slidably mounted to the first side and a second pair of sliders is slidably mounted to the second side.

20. The medical device of claim 12, wherein the plurality of control wires comprises four and the four control wires are attached to the controllable portion at 90-degree locations around a circumference of the hollow flexible shaft, where the actuator is adapted to linearly move each individual control wire independently of the other three control wires.
